(19) United States
(12) Patent Application Publication
(10) Pub. No.: US 2014/0178447 A1
(43) Pub. Date: Jun. 26, 2014

(54) REDUCTION OF BIOFILMS ON MEDICAL DEVICES

(71) Applicant: The Trustees of Columbia University in the City of New York, New York, NY (US)

(72) Inventors: Shanta M. Modak, River Edge, NJ (US); Ronald Citron, Blandon, PA (US); Santoshkumar Dongre, New York, NY (US); Nayana Baiju, Kochi (IN); Arnab Kumar Ghosh, Jersey City, NJ (US)

(21) Appl. No.: 14/194,381

(22) Filed: Feb. 28, 2014

Related U.S. Application Data
(63) Continuation of application No. PCT/US2012/052793, filed on Aug. 29, 2012.
(60) Provisional application No. 61/529,661, filed on Aug. 31, 2011.
(60) Provisional application No. 61/529,703, filed on Aug. 31, 2011.

(51) Int. Cl. A61K 31/155; A61K 31/12; A61K 31/618; A61K 31/047; A61K 31/09; A61K 31/235; A61K 31/4375; A61K 31/4025; A61K 31/05; A61K 31/555

(52) U.S. Cl. CPC A61K 31/155; A61K 31/05; A61K 31/12; A61K 31/618; A61K 31/555; A61K 31/09; A61K 31/235; A61K 31/4375; A61K 31/4025; A61K 31/047
USPC 424/407; 514/635; 514/162; 514/184; 514/544; 514/280; 514/422; 424/411

(57) ABSTRACT

Disclosed herein are methods and compositions for rendering medical devices and other surfaces biofilm resistant by the use of a coating composition comprising a combination of one or more antimicrobial agent, one or more anti-inflammatory agent, optionally a releasing agent, optionally decandediol, and a lubricious matrix system comprising a biomedical polymer. In certain embodiments said coating composition is adhered to the device or surface by a coating comprising urethane and silicone adhesives.

### PRIORITY CLAIM

This application claims priority to U.S. Provisional Applications Nos. 61/529,661 and 61/529,703, both filed on Aug. 31, 2011, both of which are incorporated by reference in their entireties herein.

### 1. INTRODUCTION

Disclosed herein are methods and compositions that inhibit the formation of biofilms on medical devices, including the biofilm-resistant devices themselves.

### 2. BACKGROUND

When a microorganism attaches to a surface, it strengthens its association with that surface by producing extracellular polymers (primarily polysaccharides). As the microorganism proliferates, or as it is joined by similar microbes, the extracellular polymers, with microorganisms embedded, constitute a structure referred to as a “Nedra”. A biofilm may be comprised of gram positive or gram negative bacteria or yeast, and may harbor only one or multiple microbe species. In the context of a biofilm, microorganisms are more prolific and much more resistant to the effects of antiseptic and antimicrobial agents compared to their unattached counterparts, and therefore constitute a significant public health problem (Donlan, 2001, Emerging Infectious Diseases 7(2):277-281; Anwar et al., 1992, Lancet 351:893-898).

### 3. SUMMARY

Disclosed herein are methods and compositions for rendering medical devices and other surfaces biofilm resistant by the application, to the device or surface, of a “lubricious coating” comprising a combination of one or more antimicrobial agent, one or more anti-inflammatory agent, optionally a releasing agent, optionally decandediol, and a lubricious matrix system comprising a biomedical polymer. In certain embodiments said lubricious coating is adhered to a device or surface by a primer coating comprising urethane and silicone adhesives.

### 4. DETAILED DESCRIPTION

For purposes of clarity and not by way of limitation, the detailed description is divided into the following subsections:

(i) biofilm microbes;

(ii) antimicrobial agents;

(iii) antiinflammatory agents;

(iv) other bioactive agents;

(v) releasing agents;

(vi) lubricious matrix system;

(vii) primer coating compositions;

(viii) one-step coating methods;

(ix) two-step coating methods;

(x) medical devices; and

(xi) specific examples.

### 4.1 Biofilm Microbes

The coating(s) disclosed herein inhibit the formation, growth and/or persistence of a biofilm on a surface. Organisms that produce such a biofilms include but are not limited to gram-positive as well as gram-negative bacteria, such as gram-positive Enterococcus faecalis, Staphylococcus aureus, Staphylococcus epidermidis, and Streptococcus viridans; and the gram-negative Escherichia coli, Klebsiella pneumoniae, Proteus mirabilis, and Pseudomonas aeruginosa, and yeast, including but not limited to Candida albicans.

### 4.2 Antimicrobial Agents

Lubricious coatings disclosed herein comprise one or more antimicrobial agent and primer coating optionally comprise one or more antimicrobial agent, which term is understood to include antibiotic agents, antiseptic agents, etc. In particular embodiments at least one such antimicrobial agent is sparingly soluble in water, for example with a solubility between 0.0008 and 30 g/L, or between 0.01 and 5 g/L, or between 0.01 and 2 g/L, at 25°. Non-limiting examples of antimicrobial agents that may be used include biguanides such as chlorhexidine (either as a free base, or a salt thereof, or a combination of free base and a chlorhexidine salt, where non-limiting examples of chlorhexidine salts include chlorhexidine diphosphanilate, chlorhexidine digluconate (also known as chlorhexidine gluconate or CHG), chlorhexidine diacetate (also known as chlorhexidine acetate or CHA), chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluoro-phosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di.alpha.-hydroxyisobutyrate, chlorhexidine diglucoheptonate, chlorhexidine di-isethionate, chlorhexidine dibenzoate, chlorhexidine dicinnamate, chlorhexidine dimandelate, chlorhexidine di-isophthalate, chlorhexidine di-2-hydroxynaphthoate, and chlorhexidine embonate), polyhexamethylene biguanide and alexidine biguanide, Triclosan®, silver salts including but not limited to silver sulfadiazine, mandelic acid, benzyl benzoate, alkanediols (in particular decanediol), povidone iodine (polyvinylpyrrolidone-iodine complex, PVI), berberine, and combinations of one or more of the foregoing agents. In non-limiting embodiments, the concentration of antimicrobial agent or agents is between about 0.1 and 5.0 percent (weight/volume) of the coating solution, or between about 0.1 and 2 percent (w/v) or between about 0.1 and 1 percent (w/v) or between about 0.1 and 0.5 percent (w/v), of the coating solution.

In particular non-limiting embodiments, the amount of chlorhexidine (free base and/or salt) in the deposited coating is between about 50-1000 micrograms per cm² or between about 250-750 micrograms per cm² or between about 500-600 micrograms per cm² and the amount of other antimicrobial agents, if present, may be between about 25-500 micrograms per cm² or between about 100-200 micrograms per cm².

### 4.3 Antiinflammatory Agents

Coatings disclosed herein comprise one or more antiinflammatory agent. In particular embodiments at least one such antiinflammatory agent is sparingly soluble in water, for example between about 0.001 and 10.0 g/L or between about 0.001 and 2 g/L at 25°. Suitable anti-inflammatory agents include but are not limited to tetrahydrocurcuminoids (THC), resveratrol, resveratrol derivatives and related compounds such as, but not limited to, trans piceid, cis piceid, trans resveratrol, cis resveratrol, pinostilbene, and pentamethoxy trans stilbene, Echinacea purpurea extract, α-terpinol, monoterpene, linalool and pinene, salicylic acid, acetyl salicylic acid (aspirin), cranberry extract and isothiocynates. In non-limiting embodiments, the concentration of antiinflammatory agent or agents is between about 0.1 and 5.0 percent (weight/volume) of the coating solution, or between about 0.1 and 2 percent (w/v) or between about 0.1 and 1 percent (w/v) or between about 0.1 and 0.5 percent (w/v). In certain non-limiting embodiments, the antiinflammatory agent is not salicylic acid or acetyl salicylic acid.

In particular non-limiting embodiments, the amount of resveratrol in a coating is about 25-500 or 100-200 or 150-200 micrograms per cm².

### 4.4 Other Bioactive Agents

In various embodiments, the coating may contain one or more additional bioactive agent, for example, but not limited to, heparin, hirudin, hydrolysing enzymes proteolytic enzyme, tissue plasminogen activator, vitamin E and/or vitamin C. In non-limiting embodiments, the concentration of bioactive agent or agents is between about 0.1 and 5.0 percent (weight/volume) of the coating solution, or between about 0.1 and 2 percent (w/v) or between about 0.1 and 1 percent (w/v) or between about 0.1 and 0.5 percent (w/v).

### 4.5 Releasing Agents

The term “releasing agent” as used herein refers to an agent that promotes the release of antimicrobial and/or antiinflammatory agent, or other bioactive agent, from the polymer matrix. Non-limiting examples include organic acids such as, but not limited to, lactic acid, glycolic acid, mandelic acid, benzoic acid, ascorbic acid and alpha hydroxy acids such as citric acid or salicylic acid. A combination of releasing agents may be used. In one specific embodiment, a combination of lactic acid and mandelic acid may be used. In non-limiting embodiments, the concentration of releasing agent is between about 0.1 and 5.0 percent (weight/volume) of the coating solution, or between about 0.1 and 2 percent (w/v) or between about 0.1 and 1 percent (w/v) or between about 0.1 and 0.5 percent (w/v) of the coating solution. In other non-limiting embodiments, the ratio of releasing agent to antimicrobial agent is between 1:0.5 and 1:10 and the ratio of releasing agent to the total amount of antimicrobial agent, antiinflammatory agent and/or additional bioactive agent is between 1:0.5 and 1:30.

### 4.6 Lubricious Matrix System

A lubricious coating as disclosed herein comprises a lubricious matrix system that comprises one or more biomedical polymer. “Lubricious” means that the matrix system provides a smooth and slippery surface which resists adherence of substances on the surface. Non-limiting examples of suitable biomedical polymers include polyurethanes of Shore hardness 70A through 72D (e.g. 93 A and 60 D), polylactic acid, polyglycolic acid, polycaprolactone, silicone polymer, silicone oil, silicone adhesive, and urethane adhesive, and combinations thereof. The adhesives may be used with or without decanediol, for example at a concentration of 0.3-5.0% w/v in the coating solution, where the addition of decanediol enhances the bonding strength) Specific non-limiting examples of urethane adhesives which may be used include from low to high viscosity Loctite Hysol® medical adhesive products, e.g. M-06FL, M-04FL, M-05 FL, M-09FL, M-11FL, M-31 Cl, M-121HP, M-4981. Non-limiting examples of silicone adhesives which may be used include Dow Corning Medical Adhesive A, Silastic Medical Adhesive Silicone Type A, Medical Adhesive MD7-4502, and Medical Adhesive MD7-4602.

In particular non-limiting embodiments, the ratio of weight of total biomedical polymer to weight of antimicrobial agent, antiinflammatory agent, other bioactive agent, or combination thereof, is between 1:1 to 10:1.

In a non-limiting embodiment, in a solution used to produce the lubricious coating, the biomedical polymer is a mixture of 93A (0.05-5.0 percent w/w) and 60D (0.05-5.0 percent w/w) polyurethanes.

In a non-limiting embodiment, a mixture of biomedical polymers is used to produce the lubricious matrix, said mixture comprising at least 50 percent or at least 60 percent or at least 70 percent or at least 80 percent or at least 90 percent of the biomedical polymers present (weight/weight).

### 4.7 Primer Coating Compositions

Also disclosed herein is a “primer” coating composition that may be applied to a device or surface that under usual conditions does not adhere well to standard coatings, for example that shows poor adherence to biomedical polyurethane coatings (for example, where the coating is not sufficiently adherent either in the initial application or under standard testing or clinical use conditions), where the primer coating renders the device or surface capable of being coated with either a standard coating or a lubricious coating as disclosed herein. In particular non-limiting embodiments, devices and surfaces in this category comprise (that is to say, at least a portion is fabricated from) a metal, for example titanium, stainless steel, or nitinol, or a silicone (e.g. a silicone polymer).

Specific non-limiting examples of urethane adhesives which may be used include from low to high viscosity Loctite Hysol® medical adhesive products, e.g. M-06FL, M-04FL, M-05 FL, M-09FL, M-11FL, M-31Cl, M-121HP, M-4981. Specific non-limiting examples of silicone adhesives which may be used include Dow Corning Silastic Medical Adhesive Type A, Medical Adhesive MD7-4502, and Medical Adhesive MD7-4602. Said “primer” coating may be used as a base coat to promote adherence of a second coating, e.g. a lubricious coating as disclosed herein, or may be used alone. Said primer coating, whether it underlies another coating layer or not, may optionally comprise one or more antimicrobial agent, one or more antiinflammatory agent, or one or more other bioactive agent, as described above.

The primer coating comprises a mixture of urethane and silicone adhesives, and optionally further comprises decanediol. The weight/weight ratio of urethane to silicone adhesive may be between about 1.25:1 to 125:1, or between about 2:1 and 20:1, or between about 2:1 and 6:1 or about 4:1, and the ratio of silicone adhesive to decanediol (if present) may be between about between 1:1 and 20:1 or between about 3:1 to 10:1 or about 5:1. In one non-limiting embodiment, the ratio of urethane adhesive:silicone adhesive:decanediol (if present) is about 20:5:1.

In one specific non-limiting embodiment a solution for creating the primer coating is as follows:

| Ingredient | % w/v | Range (% w/v) |
|---|---|---|
| Hysol ® urethane adhesive | 20.0 | 5-25 |
| Silicone Medical Adhesive | 5.0 | 0.2-20 |
| Decanediol | 1.0 | 0.1-2.0 |
| Tetrahydrofuran | 74.0 | 10.0-80.0 |

After application, said coating may be allowed to dry (for example, until detectable solvent has evaporated or for at least about 2-3 hours) prior to use either as sole coating agent or as a base for subsequently applied coating(s). In a specific non-limiting embodiment the above solution may be used to coat a urinary catheter. Note that in this and similar tables, the column “% w/v” indicates a specific concentration and the column “Range (% w/v)” shows a range that may be used.

### 4.8 One-Step Coating Methods

A coating as described above may be applied to a medical device or surface by a one-step method. Use of the term “one-step method” does not mean that it is not possible to apply one or more further coatings to the device or surface, but rather that a coating having the ingredients and properties set forth herein may be achieved in one step.

According to a first embodiment, a device or surface is coated with a solution comprising antimicrobial agent, antiinflammatory agent, releasing agent, biomedical polyurethane, and a solvent mixture comprising between 5 and 50 percent methanol and between 10 and 80 percent tetrahydrofuran (THF) and then dried at room temperature.

According to a second embodiment, a device or surface is coated with a solution comprising antimicrobial agent, antiinflammatory agent, releasing agent, biomedical polyurethane, silicone adhesive or silicone polymer, and a solvent mixture comprising between 5 and 50 percent methanol and between 10 and 80 percent THF and then dried at room temperature. The silicone renders the device more lubricious and therefore it further enhances the efficacy of catheters in preventing bacterial biofilm.

According to a third embodiment, a device or surface is coated in a solution comprising antimicrobial agent, antiinflammatory agent, releasing agent, biomedical polyurethane, urethane adhesive, silicone adhesive and/or silicone polymer, and a solvent mixture comprising between 5 and 50 percent methanol and between 10 and 80 percent THF and then dried at room temperature.

In a specific non-limiting embodiment a solution for creating the one step coating is as follows:

| Ingredient | % w/v | Range (% w/v) |
|---|---|---|
| Chlorhexidine free base | 2.0 | 0.1-5.0 |
| Lactic acid | 1.0 | 0.1-2.0 |
| Anti inflammatory agent | 1.0 | 0.1-5.0 |
| Ascorbic acid | 0.5 | 0.1-5.0 |
| PU 93A | 2.5 | 0.2-5.0 |
| PU 60D | 0.5 | 0.1-2.0 |
| Hysol ® urethane adhesive | 5.0 | 0.5-20.0 |
| Silicone adhesive | 1.0 | 0.2-20.0 |
| Tetrahydrofuran (THF) | 71.5. | 10-80 |
| Methanol | 15.0 | 5.0-50 |

In a specific non-limiting embodiment the above solution may be used to coat a urinary catheter.

In another specific non-limiting embodiment, a solution for producing the one-step coating is as follows:

| Ingredient | % w/v | Range (% w/v) |
|---|---|---|
| biguanide | 3.0 | 0.1-5.0 |
| Lactic acid | 1.0 | 0.1-2.0 |
| Anti inflammatory agent | 1.0 | 0.1-5.0 |
| PU 93A | 4.0 | 0.1-5.0 |
| PU 60D | 1.0 | 0.1-2.0 |
| THF | 60.0 | 10.0-80.0 |
| Methanol | 30.0 | 5.0-50.0 |

where the biguanide is selected from the group consisting of chlorhexidine free base, chlorhexidine acetate, or combinations thereof and the antiinflammatory is selected from the group consisting of resveratrol, Echinacea purpurea extract (EPE), and tetrahydrocurcuminoids (THC), and the biguanide activity may optionally be supplemented with one or more of the following agents (e.g. at a concentration of 0.1-2.0%) where the amount of tetrahydrofuran (THF) is adjusted in the coating solution to result in 100%: AgSD, Triclosan® (T), benzyl benzoate (BB), berberine (BR), and/or povidone iodine (PVI). As a related embodiment, up to 2 percent Silastic Medical adhesive A may be added to the above solution and the amount of methanol may be decreased accordingly to compensate for its addition (result in 100 percent).

In another non-limiting embodiment, the following coating solution may be used:

| Ingredient | % w/v | Range (% w/v) |
|---|---|---|
| CHX^{x} | 0.3 | 0.1-5.0 |
| Lactic acid | 0.3 | 0.1-2.0 |
| Anti inflammatory agent | 0.5 | 0.1-5.0 |
| Polycaprolactone | 0.5 | 0.1-5.0 |
| THF | 60.0 | 10.0-80.0 |
| Methanol | 38.4 | 5.0-50.0 |

wherein and the antiinflammatory is selected from the group consisting of resveratrol, Echinacea purpurea extract (EPE), and tetrahydrocurcuminoids (THC), and the biguanide activity may optionally be supplemented with one or more of the following agents (e.g. at a concentration of 0.1-2.0%) where the amount of THF is adjusted in the coating solution to result in 100%: AgSD, Triclosan® (T), benzyl benzoate (BB), berberine (BR), and/or povidone iodine (PVI).

Specific examples of coating solutions and methods set forth in the working example sections below are incorporated by reference into this section.

### 4.9 Two-Step Coating Methods

The methods described in this section may be used to apply, to a device or surface, a first layer of coating which is a primer coating and a second layer of coating (over at least a portion of the first layer) which is a lubricious coating. In certain non-limiting embodiments, the methods may be used to coat devices that are fabricated from silicone or metal or that comprise one or more silicone or metal surface or area, among other substrates to which conventional biomedical polyurethane does not adequately adhere, although the scope of the disclosure also extends to coating devices or surfaces that could be satisfactorily coated with conventional biomedical polyurethane.

Certain non-limiting embodiments provide a method of coating a device or surface comprising (i) coating the device or surface with a first solution comprising a urethane adhesive and a silicone adhesive, optionally comprising decanediol, dissolved in THF, to produce a primer coating layer; (ii) drying the primer coating layer at room temperature (63-73 degrees Fahrenheit) until no detectable solvent is remaining or for at least about three hours; (iii) applying, over at least a portion of the primer coating layer, a lubricious coating layer, formed by a second solution comprising antimicrobial agent, antiinflammatory agent, releasing agent, biomedical polyurethane, and a solvent mixture comprising between 5 and 50 percent methanol and between 10 and 80 percent tetrahydrofuran (THF); and (iii) drying the lubricious coating layer at room temperature.

Certain non-limiting embodiments provide a method of coating a device or surface comprising (i) coating the device or surface with a first solution comprising a urethane adhesive and a silicone adhesive, optionally comprising decanediol, dissolved in THF, to produce a primer coating layer; (ii) drying the primer coating layer at room temperature (63-73 degrees Fahrenheit) until no detectable solvent is remaining or for at least about three hours; (iii) applying, over at least a portion of the primer coating layer, a lubricious coating layer, formed by a second solution comprising antimicrobial agent, antiinflammatory agent, releasing agent, biomedical polyurethane, silicone adhesive or silicone polymer, and a solvent mixture comprising between 5 and 50 percent methanol and between 10 and 80 percent THF; and (iv) drying the lubricious coating layer at room temperature.

In one specific, non-limiting embodiment, a method of inhibiting biofilm on a medical device or surface comprises: (i) applying a first solution to the device or surface, said first solution comprising urethane adhesive (5-25 percent w/v), silastic medical adhesive silicone Type A (2-20 percent w/v), decanediol (0.1-2.0 percent w/v) in a solvent selected from the group consisting of an alcohol (e.g ethanol, methanol, isopropanol and mixtures thereof), tetrahydrofuran and mixtures thereof; (ii) allowing the first solution to dry; and (iii) applying a second solution to the device or surface, said solution comprising a biguanide (0.1-5.0 percent w/v), an anti-inflammatory agent (0.1-5.0 percent w/v), an additional antimicrobial agent (0.1-5.0 percent w/v,) polyurethane polymer at a concentration of 0.1-10 percent w/v, a solvent selected from the group consisting of an alcohol (e.g ethanol, methanol, isopropanol and mixtures thereof), tetrahydrofuran and mixture thereof and optionally a releasing agent (0.1-5.0 percent w/v).

In another specific non-limiting embodiment, a device or surface may be coated using the following protocol:

A first (primer) coating may be applied using a first coating solution as follows:

| Ingredient | % w/v | Range (% w/v) |
|---|---|---|
| Hysol ® urethane adhesive | 20.0 | 5-25 |
| Silicone Medical Adhesive | 5.0 | 0.2-20 |
| Decanediol | 1.0 | 0.1-2.0 |
| Tetrahydrofuran | 74.0 | 10.0-80.0 |

After applying the first coating, the device or surface is allowed to dry for at least 2-5 or about 3 hours, and then, onto the device or surface having the first coating, a second coating is applied using a second coating solution, as follows (METHOD A):

| Ingredient | % w/v | Range (% w/v) |
|---|---|---|
| Chlorhexidine free base | 2.0 | 0.1-5.0 |
| Lactic acid | 1.0 | 0.1-2.0 |
| Anti-inflammatory agent | 1.0 | 0.1-5.0 |
| Ascorbic acid | 0.5 | 0-5.0 |
| PU 93A | 2.0 | 0.2-5.0 |
| PU 60D | 0.5 | 0.1-2.0 |
| THF | 78.0 | 10.0-80.0 |
| Methanol | 15.0 | 0-50.0 |

In particular subsets of embodiments, the antiinflammatory agent is selected from the group consisting of resveratrol, Echinacea purpurea extract, and tetrahydrocurcuminoids and/or an additional antimicrobial agent may be included selected from the group consisting of silver sulfadiazine, Triclosan®, mandelic acid, benzyl benzoate, berberine, and povidone iodine, for example at a concentration of between about 0.1 and 2 percent (w/v). After the second coating is applied it may be allowed to dry for at least about 24-48 hours. In a specific non-limiting embodiment the above solution may be used to coat a urinary catheter.

In another specific non-limiting embodiment, a device or surface may be coated using the following protocol:

A first (primer) coating may be applied using a first coating solution as follows:

| Ingredient | % w/v | Range (% w/v) |
|---|---|---|
| Hysol ® urethane. adhesive | 20.0 | 5-25 |
| Silicone Medical Adhesive | 5.0 | 0.2-20 |
| Decanediol | 1.0 | 0.1-2.0 |
| Tetrahydrofuran | 74.0 | 10.0-80.0 |

After applying the first coating, the device or surface is allowed to dry for at least 2-5 or about 3 hours, and then, onto the device or surface having the first coating, a second coating is applied using a second coating solution, as follows (METHOD B)

| Ingredient | % w/v | Range (% w/v) |
|---|---|---|
| Chlorhexidine free base | 2.0 | 0.1-5.0 |
| Lactic acid | 1.0 | 0.1-2.0 |
| Anti-inflammatory agent | 1.0 | 0.1-5.0 |
| Ascorbic acid | 0.5 | 0-5.0 |
| PU 93A | 2.0 | 0.2-5.0 |
| PU 60D | 0.5 | 0.1-2.0 |
| THF | 76.0 | 10.0-80.0 |
| Methanol | 15.0 | 5.0-50.0 |
| Silastic Medical adhesive A | 2.0 | 1-5.0 |

In particular subsets of embodiments, the antiinflammatory agent is selected from the group consisting of resveratrol, Echinacea purpurea extract, and tetrahydrocurcuminoids and/or an additional antimicrobial agent may be included selected from the group consisting of silver sulfadiazine, Triclosan®, mandelic acid, benzyl benzoate, berberine, and povidone iodine, for example at a concentration of between about 0.1 and 2 percent (w/v). After the second coating is applied it may be allowed to dry for at least about 24-48 hours. In a specific non-limiting embodiment the above solution may be used to coat a urinary catheter.

### 4.10 Medical Devices

Medical devices and other surfaces to which the coating may be applied include but are not limited to devices and surfaces fabricated from polymers such as polyurethane, polyvinyl chloride (PVC), acrylonitrile butadiene styrene (ABS), acetals, polycarbonates, pebax, blends and alloy and block polymers; PTFE, dacron, rubber substrates such as silicone rubber, natural rubber latex, neoprene, isoprene, santoprene, blends; materials such as cotton, rayon, dacron, Spandex, woven and non-woven; and metals such as titanium, stainless steel, nitinol.

A medical device may be coated in its entirety or a single surface or portion thereof may be coated.

Surfaces that are part of an object not traditionally thought of as a medical device may also be coated, for example, but not limited to, a table or shelf surface or container.

Medical devices to which a coating may be applied (to the entire device or a portion thereof) include, for example and not by limitation, indwelling medical devices such as catheters including urinary catheters and vascular catheters (e.g. peripheral and central vascular arterial and venous catheters), wound drainage tubes, arterial grafts, soft tissue patches, gloves, shunts, stents, tracheal catheters, wound dressings, bandages, drapes, intrauterine devices, intravaginal devices, sutures, staples, guide wires and prosthetic devices (e.g. heart valves and LVADs), contact lenses, needleless connectors, endotracheal tubes, mechanical heart valves, pacemakers, peritoneal dialysis catheters, prosthetic joints, tympanostomy tubes and voice prostheses.

In various non-limiting embodiments, a device or surface set forth above is coated with a lubricious coating as described herein and/or a primer coating as described herein.

### 4.11 Specific Examples

Specific non-limiting embodiments are described in this section. In certain embodiments, a range of concentrations or amounts is set forth as well as a non-limiting example of a specific percentage; in these instances, the use of concentrations within the bounds of the ranges set forth as well as the specific percentages are contemplated.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine free base | 2.0 |
| Ascorbic acid | 0.5 |
| PU 93A | 2.0 |
| PU 60D | 0.5 |
| THF | 80.0 |
| Methanol | 15.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine acetate | 2.0 |
| Ascorbic acid | 0.5 |
| PU 93A | 2.0 |
| PU 60D | 0.5 |
| THF | 80.0 |
| Methanol | 15.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine free base | 2.0 |
| Lactic acid | 1.0 |
| Ascorbic acid | 0.5 |
| PU 93A | 2.0 |
| PU 60D | 0.5 |
| THF | 80.0 |
| Methanol | 14.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine free base | 2.0 |
| Lactic acid | 1.0 |
| Resveratrol | 1.0 |
| Ascorbic acid | 0.5 |
| PU 93A | 2.0 |
| PU 60D | 0.5 |
| THF | 78.0 |
| Methanol | 15.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine free base | 2.0 |
| Lactic acid | 1.0 |
| Tetrahydrocurcuminoids(THC) | 1.0 |
| Ascorbic acid | 0.5 |
| PU 93A | 2.0 |
| PU 60D | 0.5 |
| THF | 78.0 |
| Methanol | 15.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine free base | 2.0 |
| Lactic acid | 1.0 |
| Aspirin | 1.0 |
| Ascorbic acid | 0.5 |
| PU 93A | 2.0 |
| PU 60D | 0.5 |
| THF | 78.0 |
| Methanol | 15.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| chlorhexidine free base | 2.0 |
| Lactic acid | 1.0 |
| Resveratrol | 1.0 |
| Ascorbic acid | 0.5 |
| PU 93A | 2.0 |
| PU 60D | 0.5 |
| THF | 78.0 |
| Methanol | 15.0 |

and then allowing it to dry, for example, for 24-48 hours at room temperature.

In one non-limiting embodiment, a silicone urinary catheter is prepared as follows:

dipping it into a first coating solution as follows:

| Ingredient | % w/v |
|---|---|
| Hysol urethane adhesive | 20.0 |
| Silicone Medical Adhesive | 5.0 |
| Decanediol | 1.0 |
| Tetrahydrofuran | 74.0 |

and after applying the first coating, drying the catheter and then dipping it into a second coating solution, as follows:

| Ingredient | % w/v |
|---|---|
| Chlorhexidine free base | 2.0 |
| Lactic acid | 1.0 |
| Resveratrol | 1.0 |
| Ascorbic acid | 0.5 |
| PU 93A | 2.0 |
| PU 60D | 0.5 |
| THF | 76.0 |
| Methanol | 15.0 |
| Silastic Medical adhesive A | 2.0 |

and then allowing it to dry, for example, for 24-48 hours at room temperature.

Certain embodiments pertain to a medical device or surface thereof having one or more coating, said one or more coating comprising a biomedical polymer, an antimicrobially effective amount of chlorhexidine free base or a chlorhexidine salt; resveratrol, a fruit acid selected from the group consisting of mandelic acid, lactic acid, and a combination thereof, and decanediol.

In one non-limiting embodiment, a silicone urinary catheter is prepared as follows:

dipping it into a coating solution as follows:

| Ingredient | % w/v |
|---|---|
| Chlorhexidine free base | 2.0 |
| Mandelic acid | 1.0 |
| Lactic acid | 1.0 |
| Resveratrol | 1.0 |
| Ascorbic acid | 0.5 |
| PU 93A | 2.5 |
| PU 60D | 0.5 |
| Hysol ® urethane adhesive | 5.0 |
| Silicone adhesive | 1.0 |
| THF | 70.5 |
| Methanol | 15.0 |

and allowing it to dry at room temperature (e.g., for at least three hours).

In one non-limiting embodiment, a polyurethane central venous catheter is coated by dipping in the following solution:

| Ingredient | % w/v |
|---|---|
| CHX | 3.0 |
| Lactic acid | 1.0 |
| Resveratrol | 1.0 |
| PU 93A | 4.0 |
| PU 60D | 1.0 |
| THF | 60.0 |
| Methanol | 30.0 |

and then allowing it to dry.

In one non-limiting embodiment, a polyurethane central venous catheter is coated by dipping in the following solution:

| Ingredient | % w/v |
|---|---|
| CHA | 3 |
| Lactic acid | 1 |
| Resveratrol | 1 |
| PU 93A | 4 |
| PU 60D | 1 |
| Silastic Medical adhesive A | 0 or 2.0 |
| THF | 60 |
| Methanol | 30 or 28 |

where CHA (chlorhexidine acetate) may optionally be supplemented with silver sulfadiazine (AgSD) or triclosan (T), and then allowing it to dry.

In one non-limiting embodiment, a polyurethane central venous catheter is coated by dipping in the following solution:

| Ingredient | % w/v |
|---|---|
| CHA | 3 |
| Lactic acid | 1 |
| Resveratrol | 1 |
| PU 93A | 4 |
| PU 60D | 1 |
| Silastic Medical adhesive A | 0 or 2.0 |
| THF | 60 |
| Methanol | 30 or 28 |

where CHA (chlorhexidine acetate) may optionally be supplemented with silver sulfadiazine (AgSD) or triclosan (T), and then allowing it to dry.

In one non-limiting embodiment, a PTFE or Dacron soft tissue patch is coated/impregnated with the following solution:

1 CHLR

| Ingredient | % w/v |
|---|---|
| CHX^{x} | 3.0 |
| Lactic acid | 0.3 |
| Anti inflammatory agent | 0.5 |
| Polycaprolactone | 0.5 |
| THF | 60.0 |
| Methanol | 35.7 |

or the above solution further containing 1% decanediol (with THF reduced to result in 100%), and then allowed to dry.

In one non-limiting embodiment, an endotracheal tube is coated using the following solution:

| Ingredient | % w/v |
|---|---|
| CHA | 3 |
| Lactic acid | 1 |
| Resveratrol | 1 |
| PU 93A | 4 |
| PU 60D | 1 |
| Silastic Medical adhesive A | 0 or 2.0 |
| THF | 60 |
| Methanol | 30 or 28 |

and then allowed to dry.

In one non-limiting embodiment, a polyurethane central venous catheter is coated by dipping in the following solution:

| Ingredient | % w/v | Range (% w/v) |
|---|---|---|
| Chlorhexidine free base (CHX) | 3.0 | 2-4 |
| Mandelic acid(M) | 1.0 | 1-2 |
| Resveratrol(R) | 1.0 | 1-2 |
| Decanediol | 1.0 | 1-2 |
| PU 93A | 4.0 | 3-5 |
| PU 60D | 1.0 | 1-2 |
| Silicone adhesive MD7-4502 | 1.0 | 1-2 |
| THF | 60 | 55-70 |
| Methanol | 28 | 27-30 |

and then allowed to dry.

In one non-limiting embodiment, a polyurethane central venous catheter is coated by dipping in the following solution:

| Ingredient | % w/v | Range (% w/v) |
|---|---|---|
| Chlorhexidine free base (CHX) | 3.0 | 2-4 |
| Mandelic acid (M) | 1.0 | 1-2 |
| Silversulfadiazine (AG) | 0.75 | 0.5-1.0 |
| Resveratrol (R) | 1.0 | 1-2 |
| Decanediol | 1.0 | 1-2 |
| PU 93A | 4.0 | 3-5 |
| PU 60D | 1.0 | 1-2 |
| Silicone adhesive MD7-4502 | 1.0 | 1-2 |
| THF | 60 | 55-70 |
| Methanol | 27.25 | 27-30 |

and then allowed to dry.

In one non-limiting embodiment, a polyurethane central venous catheter is coated by dipping in the following solution:

| Ingredient | % w/v | Range (% w/v) |
|---|---|---|
| Chlorhexidine free base (CHX) | 3.0 | 2-4 |
| Mandelic acid (M) | 1.0 | 1-2 |
| Triclosan | 0.75 | 0.5-1.0 |
| Resveratrol (R) | 1.0 | 1-2 |
| Decanediol | 1.0 | 1-2 |
| PU 93A | 4.0 | 3-5 |
| PU 60D | 1.0 | 1-2 |
| Silicone adhesive MD7-4502 | 1.0 | 1-2 |
| THF | 60 | 55-70 |
| Methanol | 27.25 | 27-30 |

and then allowed to dry.

In one non-limiting embodiment, a silicone urinary catheter is prepared as follows:

dipping it into a first coating solution as follows:

| Ingredient | % w/v | Range (% w/v) |
|---|---|---|
| Hysol ® urethane adhesive | 20.0 | 5-25 |
| Silicone Medical Adhesive | 5.0 | 0.2-20 |
| Decanediol | 1.0 | 0.1-2.0 |
| Tetrahydrofuran | 74.0 | 10.0-80.0 |

and after applying the first coating, drying the catheter and then dipping it into a second coating solution, as follows:

| Ingredient | % w/v | Range (% w/v) |
|---|---|---|
| Chlorhexidine free base (CHX) | 2.0 | 1-3 |
| Mandelic acid (M) | 1.0 | 0.5-2 |
| Lactic acid | 1.0 | 0.5-2.0 |
| Silversulfadiazine(AG)/Triclosan (T) | 0.75 | 0-1.0 |
| Resveratrol (R) | 1.0 | 1-2 |
| PU 93A | 2.0 | 1-3 |
| PU 60D | 0.2 | 0.1-1.0 |
| Decanediol | 2.0 | 0.5-4.0 |
| Silicon Medical adhesive | 1.0 | 0.5-3.0 |
| THF | 72 | 30-80 |
| Methanol | 17.05 | 10-30 |

and then allowing it to dry, for example, for 24-48 hours at room temperature.

In one non-limiting embodiment, a silicone urinary catheter is prepared as follows:

dipping it into a first coating solution as follows:

| Ingredient | % w/v | Range (% w/v) |
|---|---|---|
| Hysol ® urethane adhesive | 20.0 | 5-25 |
| Silicone Medical Adhesive | 5.0 | 0.2-20 |
| Decanediol | 1.0 | 0.1-2.0 |
| Tetrahydrofuran | 74.0 | 10.0-80.0 |

and after applying the first coating, drying the catheter and then dipping it into a second coating solution, as follows:

| Ingredient | % w/v | Range (% w/v) |
|---|---|---|
| Chlorhexidine free base (CHX) | 2.0 | 1-3 |
| Mandelic acid (M) | 2.0 | 0.5-3 |
| Silversulfadiazine (AG)/Triclosan (T) | 0.75 | 0.5-1.0 |
| Resveratrol (R) | 1.0 | 1-2 |
| PU 93A | 2.0 | 1-3 |
| PU 60D | 0.2 | 0.1-1.0 |
| Decanediol | 2.0 | 0.5-4.0 |
| Silicon Medical adhesive | 1.0 | 0.5-3.0 |
| THF | 72 | 30-80 |
| Methanol | 17.05 | 10-30 |

and then allowing it to dry, for example, for 24-48 hours at room temperature.

In one non-limiting embodiment, an endotracheal tube is prepared by coating the inside, outside, or both inside and outside of the tube, or subregion thereof, with the following coating solution:

| Ingredient | % w/v | Range (% w/v) |
|---|---|---|
| Chlorhexidine free base (CHX) | 0.5 | 0.2-2.0 |
| Mandelic acid(M) | 1.0 | 0.5-2.0 |
| Silver salt(s) (e.g. silver sulfadiazine | 0.5 | 0.0-1.0 |
| and/or silver carbonate) | | |
| Resveratrol(R) | 2.0 | 0.5-3.0 |
| Decanediol | 1.0 | 0.5-3.0 |
| PU 93A | 4.0 | 2.0-5.0 |
| PU 60D | 1.0 | 0.05-2.0 |
| Silicone adhesive MD7-4502 | 1.0 | 1.0-2.0 |
| THF | 70.0 | 30.0-80.0 |
| Methanol | 19.0 | 10.0-40.0 |

and then allowing it to dry.

In certain non-limiting embodiments, an endotracheal tube is prepared by coating the inside, outside, or both inside and outside of the tube, or subregion thereof, with coating solution 1, 2 or 3 as set forth below:

Solution

| | 1 | 2 | 3 |
|---|---|---|---|
| Ingredient | % w/v | % w/v | % w/v |
| Chlorhexidine free base (CHX) | 0 | 0.5 | 0.5 |
| Mandelic acid(M) | 1.0 | 1.0 | 1.0 |
| Silver carbonate | 0.2 | 0 | 0.2 |
| Silver sulphadiazine | 0 | 0 | 0 |
| Resveratrol(R) | 2.0 | 2.0 | 2.0 |
| Decanediol | 1.0 | 1.0 | 1.0 |
| PU 93A | 4.0 | 4.0 | 4.0 |
| PU 60D | 1.0 | 1.0 | 1.0 |
| Silicone adhesive MD7-4502 | 1.0 | 1.0 | 1.0 |
| THF | 70.0 | 70.0 | 70.0 |
| Methanol | 19.8 | 19.5 | 19.3 |

and then allowed to dry.

### 5. Working Example 1

Segments of Silicone urinary catheters were coated using a two-step method to test the antimicrobial effectiveness of chlorhexidine free base (GROUP A), chlorhexidine acetate (GROUP B), or chlorhexidine free base plus lactic acid (GROUP C), as follows.

Group A

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine free base | 2.0 |
| Ascorbic acid | 0.5 |
| PU 93A | 2.0 |
| PU 60D | 0.5 |
| THF | 80.0 |
| Methanol | 15.0 |

and then allowed to dry for 24-48 hours at room temperature.

Group B

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine acetate | 2.0 |
| Ascorbic acid | 0.5 |
| PU 93A | 2.0 |
| PU 60D | 0.5 |
| THF | 80.0 |
| Methanol | 15.0 |

and then allowed to dry for 24-48 hours at room temperature.

Group C

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine free base | 2.0 |
| Lactic acid | 1.0 |
| Ascorbic acid | 0.5 |
| PU 93A | 2.0 |
| PU 60D | 0.5 |
| THF | 80.0 |
| Methanol | 14.0 |

and then allowed to dry for 24-48 hours at room temperature.

Catheter segments treated according to Group A, B and C were then tested for their zone of inhibition on a lawn of Pseudomonas aeruginosa, and the results are shown in TABLE 1.

TABLE 1
Effect of Releasing Agent on the antimicrobial
Activity: Zone of Inhibition (mm)
Test Organism: P. aeuroginosa

Days

| | | | | |
|---|---|---|---|---|
| GROUP | Drug % w/v | 1 | 2 | 3 |

| | | | | | |
|---|---|---|---|---|---|
| A | CHX | 12.0 | 0.0 | — | — |
| B | CHA | 10.0 | 9.0 | 6.0 | 5.0 |
| C | CHX + lactic acid | 12.0 | 10.0 | 8.0 | 0 |

Conclusion: The Lactic acid enhances the release of chlorhexidine free base (CHX).

### 6. Working Example 2

Segments of silicone urinary catheters were coated using a two-step method to test the antimicrobial effectiveness of chlorhexidine free base plus lactic acid combined with, as antiinflammatory agent, resveratrol (GROUP D), tetrahydrocurcuminoids (GROUP E), or acetyl salicylic acid (aspirin) (GROUP F), as follows.

Group D:

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine free base | 2.0 |
| Lactic acid | 1.0 |
| Resveratrol | 1.0 |
| Ascorbic acid | 0.5 |
| PU 93A | 2.0 |
| PU 60D | 0.5 |
| THF | 78.0 |
| Methanol | 15.0 |

and then allowed to dry for 24-48 hours at room temperature.

Group E:

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine free base | 2.0 |
| Lactic acid | 1.0 |
| Tetrahydrocurcuminoids(THC) | 1.0 |
| Ascorbic acid | 0.5 |
| PU 93A | 2.0 |
| PU 60D | 0.5 |
| THF | 78.0 |
| Methanol | 15.0 |

and then allowed to dry for 24-48 hours at room temperature.

Group F

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine free base | 2.0 |
| Lactic acid | 1.0 |
| Aspirin | 1.0 |
| Ascorbic acid | 0.5 |
| PU 93A | 2.0 |
| PU 60D | 0.5 |
| THF | 78.0 |
| Methanol | 15.0 |

and then allowed to dry for 24-48 hours at room temperature.

Catheter segments treated according to Group D, E and F were then tested for their zone of inhibition on a lawn of Pseudomonas aeruginosa, and the results are shown in TABLE 2.

TABLE 2
Effect of Anti-Inflammatory Drugs on the Antimicrobial Activity: Zone of
Inhibition (mm). CHX and LA are 2.0% and 1.0% respectively in all tests.
Test Organism: P. aeruginosa

Days

| | | | | |
|---|---|---|---|---|
| GROUP | Drug | 1 | 2 | 3 |

| | | | | | |
|---|---|---|---|---|---|
| D | Resveratrol | 15 | 12.0 | 10.0 | 5.0 |
| E | THC | 9.5 | 9.5 | 5.0 | 0 |
| F | Aspirin | 13.0 | 10.0 | 8.0 | 0 |

Conclusion: Effect of different anti-inflammatory agents on the antimicrobial efficacy of CHX and LA was investigated. The anti-inflammatory agents, especially resveratrol, were found to enhance the antimicrobial activity.

### 7. Working Example 3

Four groups of silicone urinary catheter segments were coated using the following method but where chlorhexidine was supplemented with either mandelic acid, 1.0% (w/v) (GROUP G), silver sulfadiazine, 0.75% (w/v) (GROUP H), Triclosan®, 0.5% (w/v)(GROUP I) or benzoyl benzoate, 1.0% (w/v) (GROUP J), where the THF was adjusted to result in 100% total ingredients.

A first (primer) coating was applied to a segment of a silicone urinary catheter by dipping it into a first coating solution as follows:

| Ingredient | % w/v |
|---|---|
| Hysol ® urethane adhesive | 20.0 |
| Silicone Medical Adhesive | 5.0 |
| Decanediol | 1.0 |
| Tetrahydrofuran | 74.0 |

After applying the first coating, the catheter segment was allowed to dry for 3 hours at room temperature, and then dipped into a second coating solution, as follows:

| Ingredient | % w/v |
|---|---|
| chlorhexidine free base | 2.0 |
| Lactic acid | 1.0 |
| Resveratrol | 1.0 |
| Ascorbic acid | 0.5 |
| PU 93A | 2.0 |
| PU 60D | 0.5 |
| THF | 78.0 |
| Methanol | 15.0 |

and then allowed to dry for 24-48 hours at room temperature.

The results as tested against Pseudomonas aeruginosa and Candida albicans are shown in TABLE 3 and TABLE 4, respectively.

TABLE 3
Effect of Addition of Other Antimicrobial Agents on the
Combination of Chlorohexidine (2.0%), Lactic Acid (1.0%) and
Resveratrol (1.0%): Zone of Inhibition (mm)
Test Organism: P. aeruginosa

| Day | | 1 | 2 | 3 | 4 |
|---|---|---|---|---|---|
| Group G | Mandelic acid | 14.0 | 12.0 | 11.0 | 7.0 |
| Group H | Silver sulfadiazine | 14.0 | 12.0 | 10.0 | 7.0 |
| Group I | Triclosan | 14.0 | 13.0 | 10.0 | 5.0 |
| Group J | Benzoyl benzoate | 15.0 | 13.0 | 9.0 | 5.0 |

Conclusion: The effect of other antimicrobial agents, such as mandelic acid, silver sulfadiazine (AgSD), Triclosan ® and benzoyl benzoate, was investigated along with the combination CHX + L + R. From the above study all supplemental antimicrobial agents enhanced the efficacy of CHX + L + R

TABLE 4
Effect of Addition of Other Antimicrobial Agents on the
Combination of Chlorohexidine (2.0%), Lactic Acid (1.0%) and
Resveratrol (1.0%): Zone of Inhibition (mm)
Test Organism: C. albicans

| Day | | 1 | 2 | 3 | 4 |
|---|---|---|---|---|---|
| Group G | Mandelic acid | 17.0 | 13.0 | 13.0 | 13.0 |
| Group H | Silver sulfadiazine | 15.0 | 13.0 | 11.0 | 10.0 |
| Group I | Triclosan | 17.0 | 12.0 | 12.0 | 10.0 |
| Group J | Benzoyl benzoate | 17.0 | 13.0 | 13.0 | 11.0 |

Conclusion: All these agents along with the CHX + L + R show enhanced antimicrobial efficacy against C. albicans.

### 8. Working Example 4

Six groups of silicone urinary catheter segments were coated using either Method A or Method B, as set forth in section 4.9 regarding two-step methods, above, but where chlorhexidine was supplemented with either mandelic acid, 1.0% (w/v) (GROUP K), silver sulfadiazine, 0.75% (w/v) (GROUP L), Triclosan®, 0.5% (w/v) (GROUP M), berberine, 1.0% (w/v) (GROUP N), PVI, 1.0% (GROUP O) or decanediol (GROUP P), where the THF was adjusted to result in 100% total ingredients.

The results as tested against Pseudomonas aeruginosa are shown in TABLE 5, where A and B refer to whether the coating was applied according to Method A or Method B.

TABLE 5
Effect of Addition of Other Antimicrobial Agents on the
Combination of Chlorohexidine (2.0%), Lactic Acid (1.0%) and
Resveratrol (1.0%): Zone of Inhibition (mm)
Test Organism: P. aeuroginosa

| | | Days | | | |
|---|---|---|---|---|---|
| GROUP | Drug | 1 | 2 | 3 | 4 |
| K | Mandelic acid | A = 14.0 | A = 12.0 | A = 11.0 | A = 7.0 |
| | | B = 14.0 | B = 13 | B = 12.0 | B = 99.0 |
| L | Silver | A = 14.0 | A = 12.0 | A = 10.0 | A = 7.0 |
| | sulfadiazine | | | | |
| M | Triclosan | A = 14.0 | A = 13.0 | A = 10.0 | A = 5.0 - |
| N | Berberine | A = 13.0 | A = 9.0 | | |
| | | B = 13.0 | B = 12.0 | | |
| O | PVI | A = 14.0 | A = 11.0 | A = 8.0 | A = 3.0 |
| | | B = 15.0 | B = 12.0 | B = 9.0 | B = 8.0 |
| P | Decanediol | A = 13.0 | A = 11.0 | A = 10.0 | B = 5.0 |
| | | B = 14.0 | B = 12.0 | B = 11.0 | |

Conclusion: All these agents enhances the efficacy of CHX + L + R. Method B appears to provide greater effectiveness.

The amounts of bioactive agents in the catheter segments were determined and found to be as follows: chlorhexidine level 500-580 ug/cm², Resveratrol 150-200 ug/cm² other antimicrobial agents 100-200 ug/cm².

### 9. Working Example 5

The antimicrobial activity of catheters prepared according to the two-step methods A or B (see section 4.9, above) were tested using an in vitro model consisting of two tubes, one of which was an open cylindrical tube with one end capped and the other end sealed with a rubber cork with a hole in the center (Tube 1). The tube was crimped from both the sides at the center. The second tube was open at one end and was used for collection of urine (Tube 2). Both the tubes were sterilized with ethylene dioxide. Catheter segments of 6 cm in length, with both the ends sealed with silicone to prevent intraluminal contamination with bacteria, were sterilized and were inserted from top end of “Tube 1” after lifting the cap aseptically and placed in the hole of the rubber cork at the end.

The sterile modified Trypticase Soy Agar was cooled to 40° C. and then poured along the sides of the tube around the catheter leaving the upper 1 cm of the catheter protruding out in the space above the agar tract, which represented the bladder. When the medium solidified in the tube, the cork at the bottom of the tube was removed gently without disturbing the agar column on the top thus exposing the lower end of the catheter. This lower end of the agar column with the catheter protruding represented the meatus and the agar surrounding the catheter simulated the urethra. This tube was then fixed on “Tube 2” to collect small amount of urine that flowed down the agar tract.

Inoculation of the meatus and determination of bacterial growth in the bladder was performed as follows. The “meatus” was inoculated daily with 20 μl of 10⁵ cfu/ml of P. aeruginosa after dismantling the collection tube (Tube 2). The “bladder” was filled daily with fresh sterile urine. The “bladder” and the “meatus” were cultured daily on TSA to determine the presence of bacterial growth.

On the day a positive “bladder” culture was found, the catheter segment was also processed for determination of bacterial colonization on the catheter surface. This was done by removing the catheter segment from the “bladder” end of the model, rinsing with saline and rolling it on a D/E agar plate followed by incubation for 24 hours at 37° C. to semi-quantitatively determine the bacterial growth on the surface of the catheter.

The results are shown in TABLE 6, where the test organism was Pseudomonas aeruginosa, CHX is chlorhexidine free base, and R is resveratrol

TABLE 6

| | | | CFU of adherent |
|---|---|---|---|
| | | Duration of Activity | bacteria when |
| | | Bladder (culture | bladder was positive |
| Group | Drugs | negative) (days) | (CFU/catheter) |
| control | catheter | A = 0 | A > 10⁶ |
| | uncoated | B = 0 | B > 10⁶ |
| control | catheter | A = 0 | A > 10⁶ |
| | coated with | B = 0 | B > 10⁶ |
| | polymer only | | |
| P | CHX, R, | A = 5 | A = 0 |
| | decanediol | B > 6 | B = 0 |
| K | CHX, R, | A = 2 | A > 10⁶ |
| | mandelic acid | B = 3 | B > 10⁶ |
| M | CHX, R, | A = 3 | A > 10³ |
| | triclosan | B = 4 | B > 10³ |
| O | CHX, R, | A = 4 | A = 0 |
| | PVI | B > 6 | B = 0 |
| N | CHX, R, | A = 4 | A > 10³ |
| | berberine | | |

Conclusion: PVI and Decanediol containing urinary catheters showed longer efficacy.

### 10. Working Example 6

Next, the assay system used in the above working example 5 was used to test catheters containing some of the same ingredients but prepared by the following method:

A first (primer) coating was applied to segments of a silicone urinary catheter may using a first coating solution as follows:

| Ingredient | % w/v |
|---|---|
| Hysol urethane adhesive | 20.0 |
| Silicone Medical Adhesive | 5.0 |
| Decanediol | 1.0 |
| Tetrahydrofuran | 74.0 |

After applying the first coating, the catheter segments were allowed to dry for 3 hours at room temperature and then a second coating was applied using a second coating solution, as follows:

| Ingredient | % w/v |
|---|---|
| Chlorhexidine free base | 2.0 |
| Lactic acid | 1.0 |
| Resveratrol | 1.0 |
| Ascorbic acid | 0.5 |
| PU 93A | 2.0 |
| PU 60D | 0.5 |
| THF | 78.0 |
| Methanol | 13.0 |
| Silastic Medical adhesive A | 2.0 |

After the second coating was applied the catheter segments were allowed to dry for about 24-48 hours at room temperature. Chlorhexidine was supplemented with either Triclosan®, 0.5% (w/v) (GROUP R) or decanediol (GROUP Q), where the THF was adjusted to result in 100% total ingredients. The results are shown in TABLE 6A.

TABLE 6A

| | | | CFU of adherent |
|---|---|---|---|
| | | Duration of Activity | bacteria when |
| | | Bladder (culture | bladder was positive |
| Group | Drugs | negative) (days) | (CFU/catheter) |
| control | catheter | A = 0 | A > 10⁶ |
| | uncoated | B = 0 | B > 10⁶ |
| control | catheter | A = 0 | A > 10⁶ |
| | coated with | B = 0 | B > 10⁶ |
| | polymer only | | |
| Q | CHX, R, | A > 20 | A = 0 |
| | decanediol | B > 25 | B = 0 |
| R | CHX, R, | A > 20 | A = 0 |
| | Triclosan | B > 25 | B = 0 |

Conclusion: Using the alternative coating method resulted in catheters with prolonged antimicrobial activity in this test system.

### 11. Working Example 7

Segments of silicone urinary catheters were coated with the following solution:

| Ingredient | % w/v |
|---|---|
| Chlorhexidine free base | 2.0 |
| Mandelic acid | 1.0 |
| Lactic acid | 1.0 |
| Resveratrol | 1.0 |
| Ascorbic acid | 0.5 |
| PU 93A | 2.5 |
| PU 60D | 0.5 |
| Hysol ® urethane adhesive | 5.0 |
| Silicone adhesive | 1.0 |
| THF | 70.5 |
| Methanol | 15.0 |

and allowed to dry at room temperature for at least three hours, and then tested for the resulting zone of inhibition over time against P. aeruginosa. The results are shown in TABLE 7. It should be noted that the coating was not as strong/firm as the two-step methods described in the above working example sections.

TABLE 7
Effect of Chlorhexidine (2%), Lactic acid (1.0%),
Mandelic acid (1.0%)and Resveratrol (1.0%) on antimicrobial
activity (zone of inhibition, mm)

Days

| Drug % w/v | 1 | 2 | 3 | 4 |
|---|---|---|---|---|
| CHX + LA + R + | 13 | 12.0 | 10.0 | 9.0 |
| mandelic acid | | | | |

Conclusion One step method C is also effective. but the coating appears to be not as strong/firm as the 2 step method

### 12. Working Example 8

### Application of the Technology to Depuy Spine Metal Implants

Stainless Steel and Titanium metal 2.0 cm long by 0.55 cm rods received from DePuy Spine. Inc., were coated with an antimicrobial composition to render them infection resistant. The coating needed to be smooth and not easily peeled off while maintaining significant efficacy of the coated rods in preventing bacterial biofilm formation and adherence.

The coating method employed in working example 6 was used, with chlorhexidine acetate substituted for chlorhexidine free base and mandelic acid (1%) used as a supplement.

Evaluation of Antimicrobial Activity

Procedure 1: Soak the rod one day in liquid culture and implant into a special agar tract media which simulates the subcutaneous tract. All rods were 2.0 Cm in length.

NOTE: Though in practice the metal will not be directly immersed in tissue fluid, but in contact with tissue that exudes fluid due to initial trauma, this immersion step is a severe condition and more correctly demonstrates the effectiveness of the coating during implantation.

Detailed description of Method 1: Immerse one 2.0 Cm rod into 0.5 ml of 10⁶ CFU/ml of a 1:1 mixture of Trypticase Soy Broth and Bovine Adult Serum (TSB/BAS) in a 5.0 ml culture tube (4 rods from each group were used for repetitive testing). Incubate at 37° C. in a shaker at 500 RPM speed for 24 hours. Remove the rod from the media and determine the bacterial counts in the media. Rinse the rod in saline 2 times (10 ml each time) and blot dry. Implant the rod in special agar tract media which simulates the subcutaneous tract allowing 0.2 Cm to protrude from the media. After 30 minutes, inoculate the insertion site around the rod with 25 μl of 10⁷ CFU/ml of S. aureus (ATCC 6538). Pull about half length of the rod (1 cm) out of the agar and then push the rod back into the agar leaving 0.2 Cm to protrude out. Incubate for the desired period at 37° C., 10 days. Determine bacterial adherence using sonication method in drug neutralizing broth.

The results are shown below in TABLE 8.

TABLE 8
Bacterial counts in soaking media and adherence
on Stainless Steel Rod Sample: Stainless Steel
Rod; 2.0 cm in length by 0.55 cm diameter

| | | | Log Reduction |
|---|---|---|---|
| | | | From control |
| | Control Counts | Test counts | counts |
| Counts in Media | 1.7 × 10⁹ | 5.1 × 10⁴ | 4.26 |
| After 24 Hours | | | |
| (CFU/ml) | | | |
| Adherence on Rod | 1.7 × 10⁴ | 2 | 4.01 |
| Using Sonication | | | |
| Method after 10 | | | |
| Days (CFU/Rod) | | | |

Conclusion:
The antimicrobial coating prevented bacterial adherence to the metal rod for at least 10 days after initial exposure to contaminated proteinaceous fluid and subsequent contact with a contaminated agar tract. The fluid surrounding the antimicrobial coated rod had a significantly lower bacterial count as compared to that of the control rod..

### 13. Working Example 9

A polyurethane central venous catheter was coated by dipping in the following solution:

| Ingredient | % w/v |
|---|---|
| CHX | 3.0 |
| Lactic acid | 1.0 |
| Resveratrol | 1.0 |
| PU 93A | 4.0 |
| PU 60D | 1.0 |
| THF | 60.0 |
| Methanol | 30.0 |

or a similar solution where the CHX was supplemented by decanediol, AgSD, or benzoyl benzoate (and the amount of THF component was adjusted to make 100%). Certain catheter segments were coated with the above solution where THC was substituted for resveratrol. The resulting catheters were found to have the following amounts of agents in their coating: Chlorhexidine level 500-580 ug/cm, Resveratrol 150-200 ug/cm other antimicrobial agents 100-200 ug/cm. The zones of inhibition were determined and are shown in TABLE 9.

TABLE 9
Zones of inhibition. Test Organism: P. aeruginosa

Days

| Drug | % w/w | 1 | 2 | 3 | 4 |
|---|---|---|---|---|---|
| CHX + LA + R | — | 9.8 | 7.8 | 6.0 | 5.0 |
| Decanediol | 0.5 | 7.0 | 7.0 | 7.0 | 6.7 |
| Decanediol | 1.0 | 10.0 | 7.3 | 6.3 | 5.0 |
| Silver sulfadiazine | 0.75 | 10.7 | 9.3 | 7.3 | 5.2 |
| THC* + | 0.5 | 9.0 | 9.0 | 6.7 | 6.7 |
| Benzoyl benzoate | 0.5 | | | | |

Conclusion: From the above results the combination of CHX + LA + R along with AgSD was found to be better for central venous catheters.

### 14. Working Example 10

A polyurethane central venous catheter was coated by dipping in the following solution:

| Ingredient | % w/v |
|---|---|
| CHA | 3 |
| Lactic acid | 1 |
| Resveratrol | 1 |
| PU 93A | 4 |
| PU 60D | 1 |
| Silastic Medical adhesive A | 0 or 2.0 |
| THF | 60 |
| Methanol | 30 or 28 |

or a similar solution where the CHA (chlorhexidine acetate) was supplemented by silver sulfadiazinc or triclosan and the amount of THF was adjusted to produce 100%. The results for different organisms are shown in TABLES 10-12.

TABLE 10
Test Organism: P. aeruginosa

| | Day 1 | Day 2 | Day 3 | Day 4 |
|---|---|---|---|---|
| CHA + L + R | A = 10.0 | A = 10.0 | A = 6.0 | A = 6.0 |
| | B = 8.0 | B = 7.5 | B = 6.5 | B = 5.0 |
| Silver sulfadiazine 0.75 | A = 10.5 | A = 7.5 | A = 5.0 | A = 4.5 |
| | B = 10.0 | B = 9.0 | B = 7.5 | B = 5.5 |
| Triclosan ® 0.75 | A = 9.0 | A = 8.0 | A = 7.0 | A = 5.0 |
| | B = 9.0 | B = 8.0 | B = 7.0 | B = 6.0 |

TABLE 11
The catheters are coated with the following agents in combination
with CHA, Resveratrol and lactic acid (CHA-R)
Test Organism: C. albicans

| | Day 1 | Day 2 | Day 3 | Day 4 |
|---|---|---|---|---|
| CHA + L + R | 11.5 | 9.5 | 8.5 | 6.0 |
| Silver sulfadiazine 0.75 | A = 11.0 | A = 10.5 | A = 8.0 | A = 6.5 |
| | B = 11.0 | B = 10.0 | B = 9.0 | B = 6.0 |
| Triclosan ® 0.75 | A = 11.0 | A = 10.0 | A = 9.0 | A = 7.0 |
| | B = 11.0 | B = 11.0 | B = 9.0 | B = 7.0 |

TABLE 12
The catheters are coated with the following agents in combination
with CHA, Resveratrol and lactic acid (CHA-R)
Test Organism: S. aureus

| | Day 1 | Day 2 | Day 3 | Day 4 |
|---|---|---|---|---|
| CHA + L + R | 13.5 | 12.0 | 11.0 | 10.0 |
| Silver sulfadiazine 0.75 | A = 11.5 | A = 12.0 | A = 11.0 | A = 8.0 |
| | B = 11.5 | B = 12.0 | B = 12.0 | B = 10.0 |
| Triclosan ® 0.75 | A = 12.0 | A = 12.0 | A = 13.0 | A = 11.0 |
| | B = 13.5 | B = 12.5 | B = 13.0 | B = 12.0 |

Conclusion: Both silver sulfadiazine and triclosan show similar efficacy when used along with CHXLR.

### 15. Working Example 11

### Efficacy of Central Venous Catheters in Invitro Blood Stream Model

Catheters were prepared by a one-step coating method as follows. A polyurethane central venous catheter was coated by dipping in the following solution:

| Ingredient | % w/v |
|---|---|
| CHA | 3 |
| Lactic acid | 1 |
| Resveratrol | 1 |
| PU 93A | 4 |
| PU 60D | 1 |
| Silastic Medical adhesive A | 0 or 2.0 |
| THF | 60 |
| Methanol | 30 or 28 |

or a similar solution where the CHA (chlorhexidine acetate) was supplemented by silver sulfadiazine (AgSD) or triclosan (T) and the amount of THF was adjusted to produce 100%, then dried at room temperature.

To test for antimicrobial activity, 3 Cm segment (3 sets from each group) of catheters, prepared as above, are soaked in a tube containing 4.0 ml of 10⁵ cfu/ml S. aureus culture in 50% Bovine serum and 50% Broth. The tubes were shaken in a rotary shaker at 37° C. After 24 hours, the soaking media was subcultured and the catheter segments were transferred to tubes containing fresh culture and again incubated for 24 hours. This process was repeated until the media subculture showed bacterial growth.

The catheter segments were removed from the tubes and blotted on tissue. They were rinsed twice in 10 ml saline (6 segments/10 ml saline) and blotted dry. 0.5 cm was then cut off from both the ends of each catheter segment. Each catheter segment was then put in 4 ml LTSB (drug inactivating medium) in a culture tube and sonicated for 20 min. 0.5 ml aliquot from each tube was then plated out on TSA plates and incubated for 24-48 hours.

The results are shown in TABLE 13.

TABLE 13
S. aureus adherence On CVC coated with various
antimicrobials along with resveratrol and lactic
acid (21 days after implantation and infection)

| | Adherence | Log₁₀ Reduction |
|---|---|---|
| Group | (log₁₀Cfu/cm) | from Control Counts |
| Control (uncoated catheters) | 5.8 | — |
| CHXRL | 1.9 | 3.9 |
| CHXRL AgSD | 1.7 | 4.1 |
| CHXRL T | 1.6 | 4.2 |
| Commercial CHA-AgSD catheter | 2.8 | 3.0 |

### 16. Working Example 12

PTFE and Dacron soft tissue patches were coated/impregnated with the following solution:

1 CHLR

| Ingredient | % w/v |
|---|---|
| CHX^{x} | 3.0 |
| Lactic acid | 0.3 |
| Anti inflammatory agent | 0.5 |
| Polycaprolactone | 0.5 |
| THF | 60.0 |
| Methanol | 35.7 |

or the above solution further containing 1% decanediol (with THF reduced to result in 100%).

Patches were prepared by soaking in the above solution and then suctioned using a vacuum pump and left for 5 minutes. The pieces were removed, dried and rinsed in water. After 24 hours the patches were tested for antimicrobial activity.

Adherence Testing Method:

4 Pieces of 1 cm² were soaked in the media containing 50% TSB+50% BAS (1 ml per 1 cm² of each piece) and placed on an orbital shaker at 37 C for 7 days. The pieces were removed, and transferred to a fresh media containing 10⁵ cfu of S. aureus/ml (1 ml/1 cm²) and incubated for 24 hours at 37° C. The pieces were removed and rinsed twice in (2 ml/1 cm²) saline) by vortexing at low speed, blotted dry and suspended in drug inactivating media (4.ml/1 piece) and sonicated. 0.5 ml.aliqouts were plated. The results are shown in TABLE 14, where “D” denotes the patches having decanediol in the coating solution.

TABLE 14

Adherence(Cfu/cm²)

| Group | PTFE | Dacron |
|---|---|---|
| Control (untreated Patch) | >10⁵ | >10⁵ |
| CHXLR | 70 | 120 |
| CHXLR-D | 10 | 35 |

Conclusion: The Group containing CHXLR and CHXLR-D showed lower adherence, but the one with decanediol was more effective

### 17. Working Example 13

Endotracheal tubes were coated using the following solution:

| Ingredient | % w/v |
|---|---|
| CHA | 3 |
| Lactic acid | 1 |
| Resveratrol | 1 |
| PU 93A | 4 |
| PU 60D | 1 |
| Silastic Medical adhesive A | 0 or 2.0 |
| THF | 60 |
| Methanol | 30 or 28 |

The following agents are also used in the above solution

### AgSD (0.75%)

### Triclosan (0.75%)

and the amount of THF was adjusted to result in 100%.
The catheters are coated with the following agents in combination with CHA, Resveratrol and lactic acid (CHA-R). The results are shown in TABLE 15.

TABLE 15
Test organism: P. aeruginosa

| | Day 1 | Day 2 | Day 3 |
|---|---|---|---|
| CHA + L + R | 20.0 | 20.0 | 18.0 |
| Silver sulfadiazine 0.75 | A = 21.0 | A = 19.0 | A = 20.0 |
| | B = 21.0 | B = 20.0 | B = 20.0 |
| Triclosan ® 0.75 | A = 19.0 | A = 19.0 | A = 20.0 |
| | B = 20.0 | B = 20.0 | B = 19.0 |

### 18. Working Example 14

Silicone urinary catheter segments were coated with a mixture of chlorhexidine free base, lactic acid, mandelic acid, and resveratrol plus either silver sulfadizaine (Group 1) or triclosan (Group 2), as follows.

Silicone urinary catheter segments were prepared as follows:

Dipping the catheter segment in a first primer coating solution as follows:

| Ingredient | % w/v | Range (% w/v) |
|---|---|---|
| Hysol ® urethane adhesive | 20.0 | 5-25 |
| Silicone Medical Adhesive | 5.0 | 0.2-20 |
| Decanediol | 1.0 | 0.1-2.0 |
| Tetrahydrofuran | 74.0 | 10.0-80.0 |

allowing this first coating to dry (for example, until detectable solvent has evaporated or for at least about 2-3 hours), and then applying a second (lubricious) coating using a solution as follows:

| Ingredient | % w/v |
|---|---|
| Chlorhexidine free base (CHX) | 2.0 |
| Mandelic acid (M) | 1.0 |
| Lactic acid (L) | 1.0 |
| Silversulfadiazine(AG)/Triclosan (T) | 0.75/0.75 |
| Resveratrol (R) | 1.0 |
| PU 93A | 2.0 |
| PU 60D | 0.2 |
| Decanediol | 2.0 |
| Silicon Medical adhesive | 1.0 |
| THF | 72 |
| Methanol | 17.05 |

and allowing the segments to dry.

The zones of inhibition of catheter segments according to Group 1 (AG) or Group 2 (T) were then determined against either P. aeruginosa or C. albicans; the results are shown below in TABLES 16 and 17, respectively.

TABLE 16

| Group | Drug | Day 1 | Day 2 | Day 3 | Day 4 |
|---|---|---|---|---|---|
| 1 | CHX + L + M + | 14.0 | 12.0 | 10.0 | 7.0 |
| | R + AG 0.75% w/v | | | | |
| 2 | CHX + L + M + | 14.0 | 13.0 | 10.0 | 7.0 |
| | R + T 0.75% w/v | | | | |

TABLE 17

| Group | Drug | Day 1 | Day 2 | Day 3 | Day 4 |
|---|---|---|---|---|---|
| 1 | CHX + L + M + | 15.0 | 13.0 | 11.0 | 10.0 |
| | R + AG 0.75% w/v | | | | |
| 2 | CHX + L + M + | 17.0 | 12.0 | 12.0 | 10.0 |
| | R + T 0.75% w/v | | | | |

### 19. Working Example 15

Segments of endotracheal tube were coated with solution 1, 2 or 3 as follows and then allowed to dry, and tested as set forth below.

Solution

| | 1 | 2 | 3 |
|---|---|---|---|
| Ingredient | % w/v | % w/v | % w/v |
| Chlorhexidine free base (CHX) | 0 | 0.5 | 0.5 |
| Mandelic acid(M) | 1.0 | 1.0 | 1.0 |
| Silver carbonate | 0.2 | 0 | 0.2 |
| Silver sulphadiazine | 0 | 0 | 0 |
| Resveratrol(R) | 2.0 | 2.0 | 2.0 |
| Decanediol | 1.0 | 1.0 | 1.0 |
| PU 93A | 4.0 | 4.0 | 4.0 |
| PU 60D | 1.0 | 1.0 | 1.0 |
| Silicone adhesive MD7-4502 | 1.0 | 1.0 | 1.0 |
| THF | 70.0 | 70.0 | 70.0 |
| Methanol | 19.8 | 19.5 | 19.3 |

The effectiveness of the coatings at preventing biofilm formation was tested using an airway model. The model consists of a 50 mL sterile culture tube containing 30 mL of a specially constituted sterile medium [1% Difco agar (Fischer Scientific Co., Atlanta, Ga., USA), 0.03% trypticase soy agar (TSB; Fischer Scientific Co.), 5% bovine adult serum (BAS; Sigma Chemicals, St Louis, Mo. USA), 0.5% whole milk UHT (Parmalat) in phosphate-buffered saline (PBS)] to simulate the endotracheal lumen. The agar column is inoculated at the top with 10 μL of the test organism, S. aureus (10⁸ cfu/mL). The endotracheal tube (ETT) segment is then pushed through the agar column from the top leaving the upper 0.5 cm of the segment protruding out of the agar tract. The proximal end of the agar column where the ETT segment is inserted is considered as the ‘mouth’ and the distal end inserted into the media as the ‘tracheal’ portion of the airway model. The tubes are incubated at 37° C. for five days, and the bacterial colonization on the surface of ETT is determined. Here, the proximal (mouth) and distal (trachea) portions were subcultured to determine the bacterial colonization. The results are shown in TABLE 18. Note that on day 5 the tubes were reinserted in fresh airway model and inoculated with 10 microliters of S. aureus. The control was an uncoated ETT segment processed similarly to coated segments as described in the method above. The results indicate that endotracheal tubes coated with solution 3 showed higher activity compared to the other coatings.

TABLE 18

| | Bacterial Colonization | | Subculture (CFU/plate, log10) | | | |
|---|---|---|---|---|---|---|
| | log₁₀ Growth | | Proximal | | Distal | |
| | 5 day | 12 day | 5 day | 12 day | 5 day | 12 day |
| Control | 4.1 | 5.2 | 5.2 | 5.5 | 5.2 | 5.5 |
| 1 | 2.85 | 4.3 | 0.6 | 3.5 | 0.6 | 3.9 |
| 2 | 1.93 | 3.5 | 0 | 3.2 | 0.0 | 3.2 |
| 3 | 1.60 | 1.5 | 0 | 1.1 | 0 | 1.2 |

Various publications are cited herein that are hereby incorporated by reference in their entireties.

1. A medical device or surface thereof having one or more coatings, said one or more coatings comprising effective amounts of an antimicrobial agent, antiinflammatory agent, and biomedical polymer.

2. The device or surface of claim 1 where the antimicrobial agent is selected from the group consisting of a biguanide, Triclosan®, silver salts, benzyl benzoate, alkanediol, povidone iodine, berberine and combinations thereof.

3. The device or surface of claim 2 where the biguanide is selected from the group consisting of chlorhexidine, PHMB, Alexidine and combinations thereof.

4. The device or surface of claim 3 where the chlorhexidine is selected from the group consisting of chlorhexidine free base, chlorhexidine acetate, chlorhexidine gluconate, and combinations thereof.

5. The device or surface of claim 1, where the one or more coatings further comprises a releasing agent selected from the group consisting of, lactic acid, glycolic acid, mandelic acid, benzoic acid, and ascorbic acid.

6. The device or surface of claim 1, where the one or more coatings further comprises an adhesive selected from the group of a silicone adhesive, a urethane adhesive, and combinations thereof.

7. The device or surface of claim 1, where the one or more coatings further comprises decanediol.

8. The device or surface of claim 1, wherein the one or more coatings further comprises a releasing agent and adhesive, wherein the releasing agent promotes release of the antimicrobial agent or antiinflammatory agent; and wherein the biomedical polymer is a polyurethane, and the adhesive is a mixture of silicone and urethane adhesives.

9-11. (canceled)

12. The device or surface of claim 1, wherein the antiinflammatory agent is selected from the group consisting of a tetrahydrocurcuminoid, resveratrol, trans piceid, cis piceid, trans resveratrol, cis resveratrol, pinostilbene, pentamethoxy trans stilbene, Echinacea purpurea extract, α-terpinol, monoterpene, linalol, pinene, cranberry extract and isothiocynate.

13. The device or surface of claim 8, where the releasing agent is selected from the group consisting of lactic acid, glycolic acid, mandelic acid, benzoic acid, and ascorbic acid.

14. The device or surface of claim 8, wherein the one or more coatings further comprises decanediol.

15. The device or surface of claim 1 wherein the antimicrobial agent comprises chlorhexidine free base or a chlorhexidine salt; the antiinflammatory agent comprises resveratrol; and wherein the one or more coatings further comprises a fruit acid selected from the group consisting of mandelic acid, lactic acid, and a combination thereof, and decanediol.

16. The device or surface of claim 15, said one or more coatings further comprising an additional antimicrobial agent selected from the group consisting of silver sulfadiazine, a silver salt, triclosan, povidone iodine and a combination thereof.

17. The device or surface of claim 15, said one or more coatings further comprising a silicone adhesive.

18. A method of reducing biofilm on a medical device or surface thereof, comprising applying one or more coatings to the device or surface, the one or more coatings comprising effective amounts of an antimicrobial agent, antiinflammatory agent, and biomedical polymer.

19. The method of claim 18 where the antimicrobial agent is selected from the group consisting of a biguanide, Triclosan®, silver salts, benzyl benzoate, alkanediol, povidone iodine, berberine and combinations thereof.

20. The method of claim 19 where the biguanide is selected from the group consisting of chlorhexidine, PHMB, Alexidine and combinations thereof.

21. The method of claim 20 where the chlorhexidine is selected from the group consisting of chlorhexidine free base, chlorhexidine acetate, chlorhexidine gluconate, and combinations thereof.

22. The method of claim 18, wherein the one or more coatings further comprises a releasing agent selected from the group consisting of, lactic acid, glycolic acid, mandelic acid, benzoic acid, and ascorbic acid.

23. The method of claim 18, wherein the one or more coatings further comprises an adhesive selected from the group of a silicone adhesive, a urethane adhesive, and combinations thereof.

24. The method of claim 18, wherein the one or more coatings further comprises decanediol.

25. The method of claim 18, wherein the one or more coatings further comprises a releasing agent and adhesive, wherein the releasing agent promotes release of the antimicrobial agent or antiinflammatory agent; and wherein the biomedical polymer is a polyurethane, and the adhesive is a mixture of silicone and urethane adhesives.

26-28. (canceled)

29. The method of claim 18, where the antiinflammatory agent is selected from the group consisting of a tetrahydrocurcuminoid, resveratrol, trans piceid, cis piceid, trans resveratrol, cis resveratrol, pinostilbene, pentamethoxy trans stilbene, Echinacea purpurea extract, α-terpinol, monoterpene, linalol, pinene, cranberry extract and isothiocynate.

30. The method of claim 18, wherein the one or more coatings further comprises a releasing agent selected from the group consisting of lactic acid, glycolic acid, mandelic acid, benzoic acid, and ascorbic acid.

31. The method of claim 18, wherein the one or more coatings further comprises decanediol.

32. The method of claim 18, wherein the antimicrobial agent comprises chlorhexidine free base or a chlorhexidine salt; the antiinflammatory agent is selected from the group consisting Echinacea purpurea extract tetrahydrocurcuminoid, resveratrol and combinations thereof; and wherein the one or more coatings further comprises a fruit acid selected from the group consisting of mandelic acid, lactic acid, and a combination thereof, and decanediol.

33. The method of claim 32, said one or more coatings further comprising an additional antimicrobial agent selected from the group consisting of silver sulfadiazine, a silver salt, triclosan, povidone iodine and a combination thereof.

34. The method of claim 32, said one or more coatings further comprising a silicone adhesive.

35. The method of claim 32, said method comprising
(i) applying, to the device or surface, a primer coating comprising an adhesive selected from the group consisting of a silicone adhesive, a urethane adhesive, and a combination thereof; decanediol and tetrahydrofuran;
(ii) allowing the primer coating to dry;
(iii) applying, over the primer coating, a lubricious coating comprising the biomedical polymer, chlorhexidine free base or chlorhexidine salt, antiinflammatory, fruit acid, and decanediol of claim 32; and
(iv) allowing the lubricious coating to dry.

36. The method of claim 35 where the lubricious coating further comprises a silicone adhesive.

37. The method of claim 32, wherein the one or more coatings comprises
(i) between about 0.1 and 5 percent (w/v) chlorhexidine free base or a chlorhexidine salt;
(ii) between about 0.1 and 2 percent (w/v) resveratrol;
(iii) between about 0.1 and 2 percent (w/v) fruit acid selected from the group consisting of mandelic acid, lactic acid, and a combination thereof;
(iv) between about 0.3 and 5.0 percent (w/v) decanediol; and
(v) further comprising an adhesive selected from the group consisting of a silicone adhesive, a urethane adhesive, and a combination thereof.

38. The method of claim 35, wherein the primer coating comprises
(i) between about 0.2 and 20 percent (w/v) of a silicone adhesive;
(ii) between about 5 and 25 percent (w/v) of a urethane adhesive;
(iii) between about 0.1 and 2.0 percent (w/v) decanediol; and
(iv) between about 10.0 and 80 percent (w/v) tetrahydrofuran,
and wherein the lubricious coating comprises
(v) between about 0.05 and 5 percent (w/v) biomedical polymer;
(vi) between about 0.1 and 5 percent (w/v) chlorhexidine free base or chlorhexidine salt;
(vii) between about 0.1 and 5 percent (w/v) antiinflammatory;
(viii) between about 0.1 and 2 percent (w/v) fruit acid;
(ix) between about 0.3 and 5 percent (w/v) decanediol.

39. A medical device prepared according to the method of claim 37.

40. The medical device of claim 39, selected from the group consisting of catheter, wound drainage tube, arterial graft, soft tissue patch, glove, shunt, stent, tracheal catheter, wound dressing, bandage, drape, intrauterine device, intravaginal device, suture, staple, guide wire, prosthetic device, contact lens, needleless connector, endotracheal tube, mechanical heart valve, pacemaker, peritoneal dialysis catheter, prosthetic joint, tympanostomy tube and voice prosthesis.

41. A medical device prepared according to the method of claim 38.

42. The medical device of claim 41, selected from the group consisting of catheter, wound drainage tube, arterial graft, soft tissue patch, glove, shunt, stent, tracheal catheter, wound dressing, bandage, drape, intrauterine device, intravaginal device, suture, staple, guide wire, prosthetic device, contact lens, needleless connector, endotracheal tube, mechanical heart valve, pacemaker, peritoneal dialysis catheter, prosthetic joint, tympanostomy tube and voice prosthesis.
